(19) United States
(12) Patent Application Publication
(10) Pub. No.: US 2016/0228293 A1
(43) Pub. Date: Aug. 11, 2016

(54) METHOD AND APPARATUS FOR CLOSING THE EYES OF PERSONS WITH NOCTURNAL LAGOPHTHALMOS

(71) Applicant: Robert Latkany, Rye, NY (US)

(72) Inventor: Robert Latkany, Rye, NY (US)

(21) Appl. No.: 14/730,300

(22) Filed: Jun. 4, 2015

Related U.S. Application Data
(60) Provisional application No. 62/114,950, filed on Feb. 11, 2015.

(51) Int. Cl. A61F 9/007; A61H 5/00

(52) U.S. Cl. CPC A61F 9/00718; A61H 5/00; A61H 2205/024

(57) ABSTRACT

This invention provides a method and apparatus for closing the eyelids of persons suffering from nocturnal lagophthalmos (the inability to close the eyelids when sleeping), so as to keep their eyes moist and to protect their eyes from contacting the bedding while they sleep. The method is to apply gentle pressure on the inner ends of the eyelids with soft fingerlike protrusions affixed to a support that spans the nose and is held in place by adjustable, elastic headbands. This pressure pulls the eyelids towards the nose, which forces the eyelids to close and stay closed, thereby preventing loss of tear fluids by evaporation. In one embodiment, pressure can also be applied to the nasolacrinal drainage system to prevent tear fluids from draining out of the eye into the nasal cavity.

### CROSS-REFERENCES TO RELATED APPLICATIONS

This application claims priority of U.S. Ser. No. 62/114,950, filed Feb. 11, 2015, the entire content of which is incorporated by reference into this application.

### FIELD OF THE INVENTION

This invention relates to the methods and means of closing the eyes of persons who cannot close their eyes completely (a condition known as lagophthalmos).

### BACKGROUND OF THE INVENTION

A normal, healthy eye is covered by tear fluids, which form a film that moistens the surface and washes away dust and other foreign materials. This film is replenished and redistributed when the eyelids close, as when blinking. Normally, tear fluids are produced continuously and the excess drains away through holes (called puncta) near the nose, which direct the fluids through a nasolacrimal drainage system into the nasal cavity. If the eyelids do not close, the film of tears evaporates, resulting in a condition known as “dry eye”. Dry eyes are uncomfortable and are also subject to injury or infection from foreign materials landing on the exposed eye surface or from abrasion resulting from lack of lubrication between the eye surface and the eyelids. The dry eyes caused by lagophthalmos can lead to permanent loss of vision.

Millions of Americans suffer from lagophthalmos. Some common causes of lagophthalmos are: facial paralysis (such as Bell's palsy); stroke; infection; trauma; skin conditions (such as ichthyosis); thyroid disorder; sand surgical procedures.

When people cannot completely close their eyes while sleeping, this condition is called nocturnal lagophthalmos. People usually don't know they have it, but their doctor may recognize the symptoms: irritation of and damage to the surface of the eye; poor sleep patterns; and/or complaints of dry and painful eyes upon awakening.

There are several methods for treating dry eye from lagophthalmos and other causes. When one who has Dry Eye condition (the Subject) is awake, eye drops and ointments can be employed to moisten and irrigate the eye and to supplement and stimulate production of natural tears that normally serve this purpose. The puncta that drain tears out of the eye can be plugged, or the nasolacrimal drainage system can be pressed shut, to retain natural tears and medications in the eye. The eyelids can be surgically altered and/or metal weights can be taped to or implanted in the upper lid to pull them shut. Goggles that have means to provide heat, cold, humidity or medication can be used for periodic treatment.

When the Subject is asleep, many of these methods are generally less effective or ineffective. e.g.: periodic application of eye drops is impractical; ointments that remain in the eye are messy; and weights no longer pull the upper eyelid down. The eyelids can also be taped shut with medical tape, but the tape is uncomfortable, can cause irritation and can pull out eyelashes.

Some sleep goggles for treating dry eyes provide a protective vault over the eye socket so that the exposed eye tissue won't be damaged by contact with bedding. Within the vault, some goggles provide moisturizing media to create a humid environment for the exposed eye tissue, which is said to relieve the dry eye condition. An example of this type of apparatus is disclosed in Davison, et al., Apparatus, system and method for treating dry eye conditions and promoting healthy eyes, U.S. Pat. No. 7,231,922 (the “Davison 922” patent). The apparatus of this patent creates a “moisture chamber” around the eye, wherein an absorbent material holds water or medicine, which is vaporized by body heat to produce humidity or medicinal vapor in the chamber. The Davison 922 patent does not provide means to close the eyelids. A foreign patent application related to the Davison 922 patent is Davison, et al., Apparatus, system and method for treating dry eye conditions and promoting healthy eyes, WO 2005/01585 A2 (the “Davison WO” patent application).

Other sleep goggles provide pads to apply pressure to the entire eyelids to hold them shut. The pads are either inserted in goggles that hold them in place or by bands that extend around the head to hold them in place. The pressure exerted by these pads distorts the eyeball and causes faulty vision for a time after they are removed. This pressure may permanently affect the eyeball, e.g., cause astigmatism. Another complaint against applying pressure directly to the eye is that the pressure pads bend the eyelashes and make them unsightly. This is similar to the complaint that when tape is used to close the eyes, the tape pulls out eyelashes. A general problem with goggles is that they can exert pressure that can damage the tissues, blood vessels and nerves that they press upon. Thus, there is a need for improved methods and means to treat nocturnal lagophthalmos.

### SUMMARY OF THE INVENTION

The present invention provides a method and means to treat nocturnal lagophthalmos by closing eyelids during sleep, so that evaporation of tear fluids is stopped. This is accomplished by applying gentle pressure to the inner end of the eyelids. The principle of its action can be demonstrated by lightly pressing a fingertip slightly downward against the inner ⅛th to ¼th of the upper eyelid. As the finger slides obliquely down the side of the eyeball it pulls the eyelid towards the nose and stretches it across the eyeball. This will close the eyelids on that eye and hold them closed. Note that the finger presses on the bone around the eye socket, not on the eyeball. This invention works the same way to pull the eyelids towards the nose.

In one embodiment, the present invention employs a support member that partially covers the eyes and nose. In one embodiment, this support member is constructed with two layers of material: an outer layer of rigid material to provide structural strength; and an inner layer of soft material that provides comfort where the support bears on the skin of the face and where the apparatus contacts the eyelids to close them. It has soft protrusions on the inner side, which are designed to press upon and close a Subject's eyelids.

In one embodiment, the support member rests on the bones at the inner ends of the eye sockets, covering each eye and is held in place by elastic bands that extend around the head. The inner layer (the side toward the face) of the support member has a protrusion on each side that extends from the support member to contact the inner end of the eyelid. In one embodiment, the protrusion is fashioned from a soft pliable material that is water resistant. The dimensions of the protrusions are selected to accommodate the size and shape of an individual's face. It must be sufficiently long so that as the support member is placed on the face and secured in place with elastic head bands, the protrusions will contact the inner ⅛th to ¼th of the upper eyelid. There it applies gentle pressure obliquely to the eyeball and pulls the eyelid towards the nose, which closes the eyelid and holds it closed.

In another embodiment, there is provided an apparatus for pulling one or both eyelids over the eyes of a subject, said apparatus comprises a pair of goggles and means to pull the eyelids over the eyes, wherein said means for pulling the eyelids is located on the side of the google that touches the subject's face, wherein when said goggle is secured on the subject's face, said means to pull the eyelids will contact the inner portion of the subject's upper eyelid(s) and apply gentle pressure obliquely to pull the eyelid(s) towards the nose, thereby pulling the eyelid(s) over the eye(s) and holding the eye(s) closed.

In one embodiment, the apparatus of the present invention can also have clamps or protrusions that apply pressure to the nasolacrimal sacs to prevent drainage of tear fluids out of the eye to further increase retention of tear fluids in the eyes.

### BRIEF DESCRIPTION OF THE DRAWINGS

FIG. 1 shows one embodiment of the apparatus over the Subject's open eyes, ready to place the elastic bands 2 around the head to hold the apparatus in the proper position. Cross section X-X shows details of the pressure protrusions 4 on the inner surface of the support member 1. 3 is the soft material on the inner surface of the support member.

FIG. 2 shows one embodiment of the apparatus in place, with the pressure protrusions 4 pulling the eyelids shut. The soft protrusions 4 yield and bend toward the nose as they pull the eyelids toward the nose.

FIG. 3 shows another embodiment of the apparatus over the Subject's open eyes. Cross section X-X shows details of the pressure protrusions 8 on the inner surface of the support member 5. In one embodiment, 7 is the soft foam liner on the inner surface of the support member, and 6 designates the elastic bands.

FIG. 4 shows one embodiment of using the apparatus of FIG. 3, with the pressure protrusions 8 pulling the eyelids shut.

FIG. 5 shows another embodiment of the apparatus of the present invention. 9: support member; 10: flexible strap over the bridge of the nose; 11: foam protrusion, or contacting member; and 12: elastic strap.

FIGS. 6A-6C show several other embodiments of the apparatus of the present invention. 13: support member or goggle; 14: flexible strap over the bridge of the nose; 15: elastic strap for wrapping around the head; 16: foam around edge of support member or goggle; 17: foam protrusion to press on eyelid; 18: bubble or sac protrusion to press on eyelid; 19: bubble or foam protrusion to press on eyelid; 20: flap valve for inflating bubble; 21: screw for extending protrusion.

### DETAILED DESCRIPTION OF THE INVENTION

The present invention provides a method and apparatus to close a Subject's eyes when the Subject is sleeping or resting his eyes. In one embodiment, the apparatus of the present invention comprises a support member that covers over the nose and eye sockets, extending about half way across the eyelid over each eye, and covers from the lower forehead to below the lower edge of the eye sockets. Adjustable elastic bands that encircle the Subject's head are attached to hold it in place. In one embodiment, this support member is made from a lightweight, rigid material on the outside, which is shaped to conform to the nose and forehead. In one embodiment, the support member rests on the bones around the central edges of the eye sockets: on the forehead at the top about at the central ends of the eyebrows, and at the bottom near the nose below the eye socket.

In one embodiment, the support member has an inner layer of foam to prevent discomfort where it rests on the face. The support member also has soft protrusions near the edges over the eyelids. When the apparatus is put in place and the elastic bands are put around the head, these protrusions contact the eyelids with a gentle inward pressure and pull the eyelids closed. Similar protrusions to occlude the nasolacrimal sacs can also be incorporated into the apparatus to make it even more effective. In one embodiment, the protrusions are made of soft medical foam or elastomer, or can be jel-filled sacs. These protrusions may be part of the inner foam layer, small pieces that are held in place with adhesive or pieces held in pockets in the support member.

The present invention fills a need for an effective, comfortable apparatus to hold a Subject's eyes closed when sleeping or resting, thereby preventing evaporation of tear fluids and keeping the eye moist. The present invention applies gentle pressure obliquely to the side of the eyeball, primarily on the tissues of the eyelids and between the eye and nose. Other methods of holding eyelids closed either use pads to apply greater pressure over a wide part of the eyelids and eyeball, which distorts the eyeball and causes visual problems, or use medical tape to hold the eyelids shut, which causes irritation and discomfort and pulls out eyelashes. In one embodiment, the present invention can also provide clamps or protrusions to occlude the nasolacrimal drainage system, which will further improve retention of fluids in the Subject's eyes.

In one embodiment, the apparatus of the present invention comprises a support member (1) covers over the nose and eye sockets, extending about half way across the eyelid over each eye, and covers from the lower forehead to below the lower edge of the eye sockets. In one embodiment, adjustable, elastic bands (2) that encircle the Subject's head are attached to hold the support member in place. The outer surface of this support member is made from a lightweight, rigid material, including but not limited to polyvinylchloride, polyethylene, polypropylene or some other suitable thermoplastic material. It is shaped to conform to the places where it contacts the Subject's face.

In one embodiment, the inner surface of the support member has soft material (3), such as medical grade foam, attached to provide comfort where it rests on the face. In one embodiment, the support member has four support points over the bones around the eye sockets: two on the forehead about at the inner ends of the eyebrows above the inner corners of the eye sockets; and two on either side of the nose below the inner corners of the eye sockets. In one embodiment, there are also two protrusions (4) on the inner surface near the edges of the support member, which are over the innermost ⅛ to ¼ inch of the upper eyelids. When the apparatus is put in place and elastic bands are put around the head, these protrusions pull the eyelids inward (toward the nose) and downward (toward the mouth) with gentle pressure, which pulls the eyelids closed. Slightly larger or separate protrusions can be used to also occlude the nasolacrimal sacs on the sides of the nose to make the apparatus even more effective at retaining tear fluids in the eye.

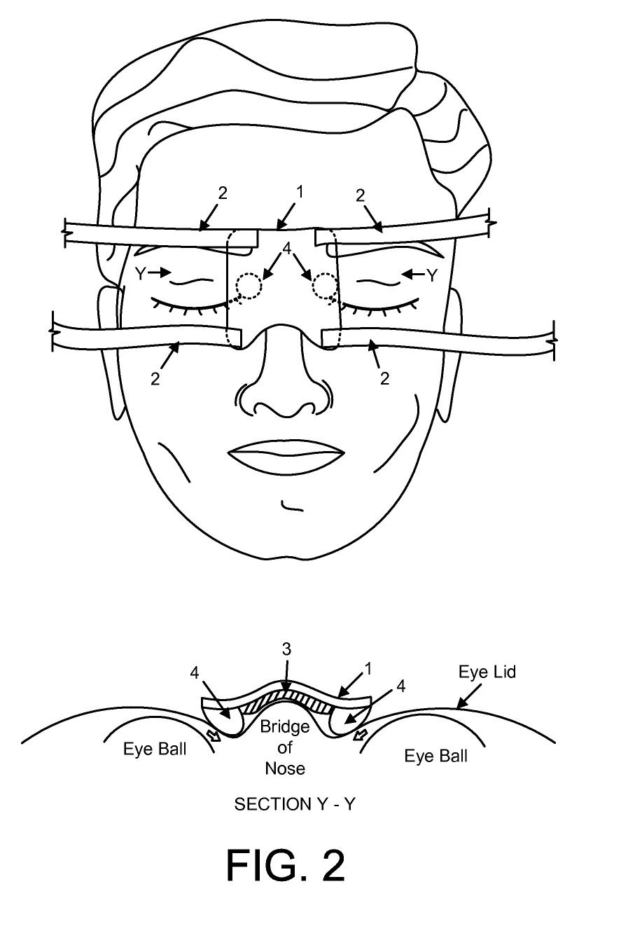

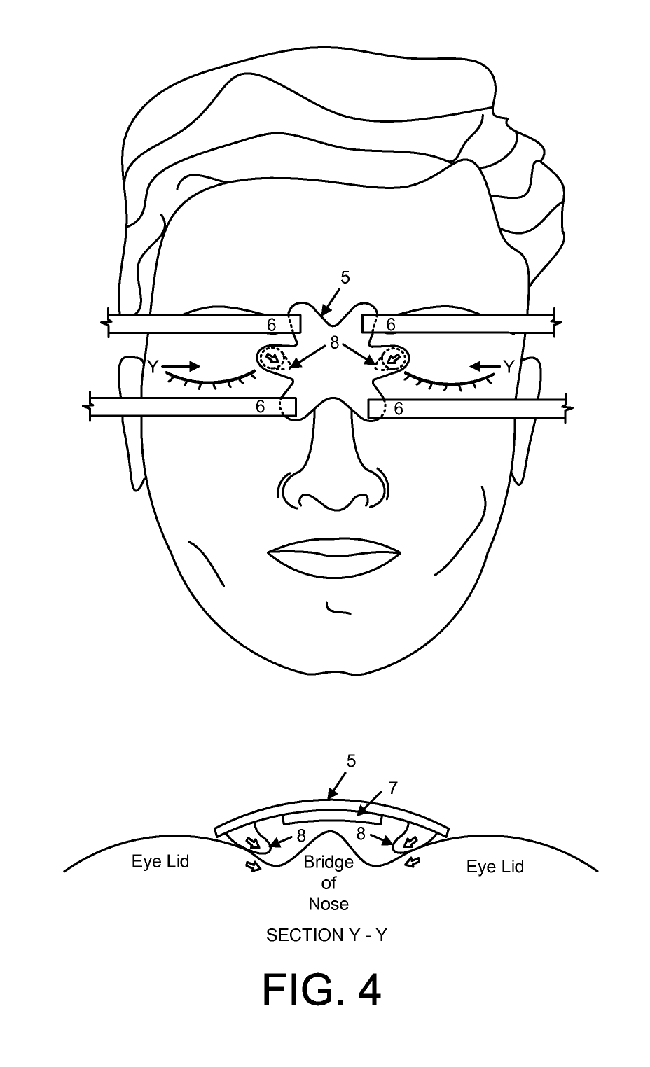

In one embodiment, the protrusions are made of soft medical foam, elastomer or gel-filled sacs. They may be molded as part of the inner foam layer, small pieces of medical foam or elastomer that are held in place with adhesive or pieces held in a pocket in the support member. The size of the protrusions depends on the design of the apparatus and the contours of the Subject's face. In particular, the length of the protrusion must exceed the distance from the edge of the support member to the inner end of the Subject's eyelid and must be long enough to draw the eyelids closed. In one embodiment, a generally satisfactory shape and size is an oblique paraboloid with a base diameter and height of about ⅜ths of an inch, which is canted about 20 to 40 degrees toward the nose and downward. The softness must be about that of a fingertip and soft enough to allow the protrusion to deform as it bends toward the nose, as shown in Section Y-Y in FIGS. 2 and 4, where the direction that the eyelids are pulled is shown with small arrows.

One of ordinary skill in the art would readily choose various materials to construct the apparatus of the present invention; for example, one may choose from those discussed in paragraphs 0053-0057 in the Davison WO publication, among others.

One skilled in the art would readily design variations from the apparatus of the foregoing paragraphs. For example, the support member can be made to flex around the bridge of the nose as it is put in place and secured by the elastic straps. This would cause the protrusions to pivot toward the nose as they move in toward the face. As a result, its movement relative to the eyeball would be more nearly tangential, the pulling action would be enhanced and pressure on the eyeball would be minimized.

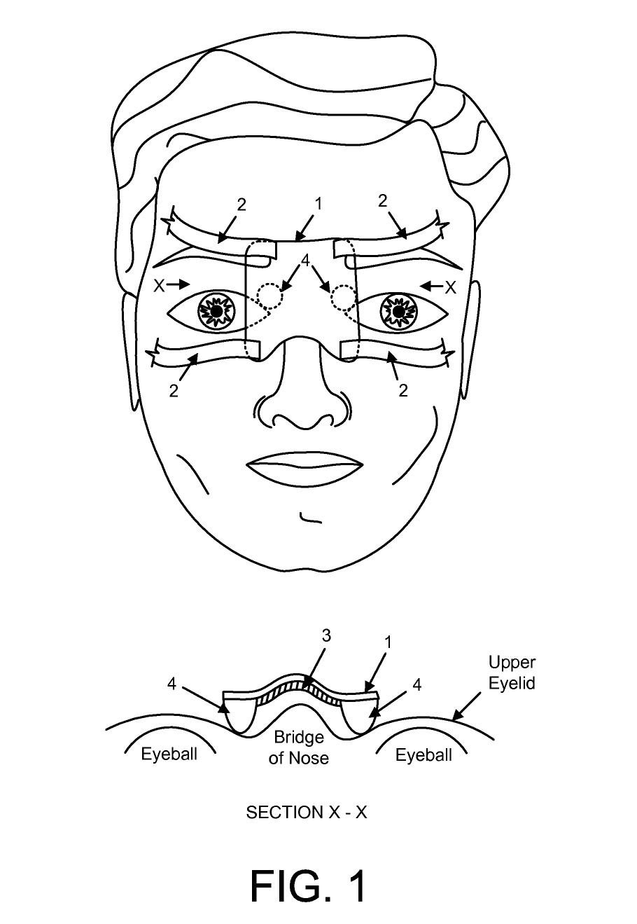

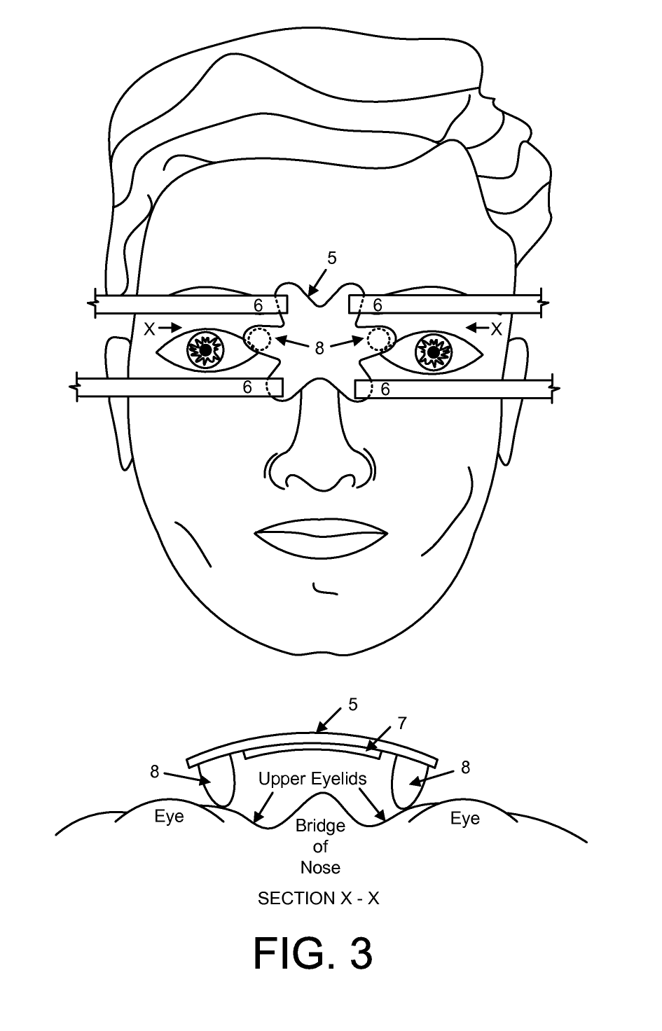

The shape of the support member may differ from that in FIGS. 1 and 2, as long as it performs the essential functions: holding the device in place; and positioning the protrusions to properly pull the eyelids closed. For example, one variation is to shape the support member like an asterisk, with six arms crossing at the bridge of the nose: four go to the four support points discussed earlier and two go out sidewise to hold the protrusions over the eyes (see FIGS. 3-4). In another embodiment, the present invention can be further modified to employ an “X” shaped support member, with no edges extending over the eyes. A bar would clip to the support member above the bridge of the nose and would extend outward over the inner end of the eyes. This bar, in turn, would have replaceable protrusions attached to the ends over the eyelids. The bar and protrusions could be selected from a selection of sizes to fit each individual's measurements.

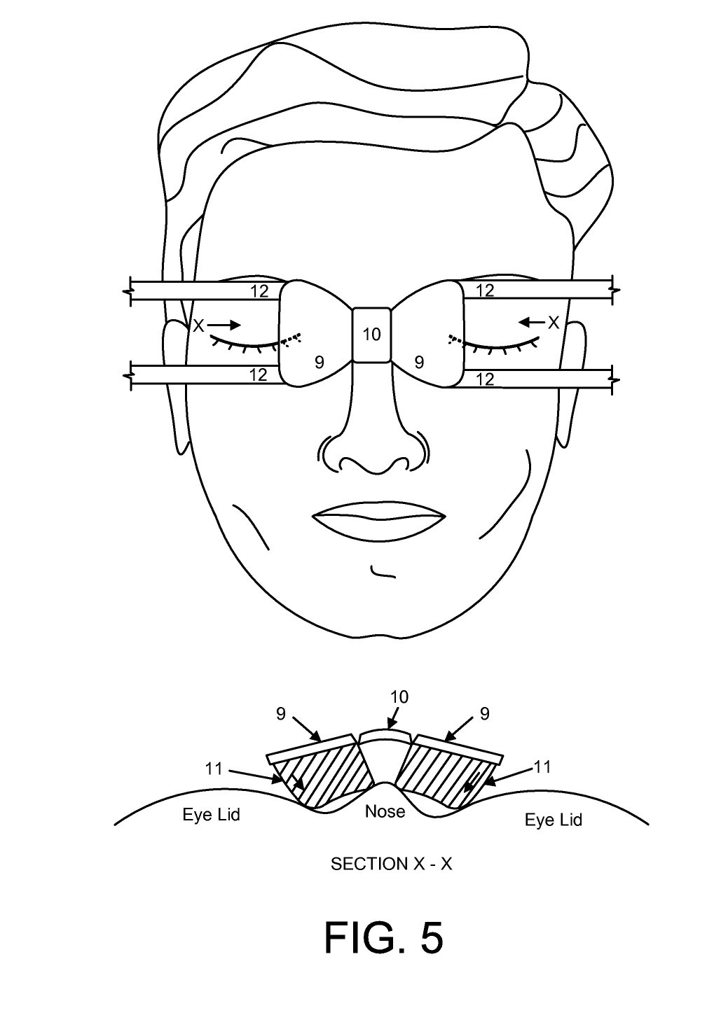

In another embodiment, the support member is in two parts connected across the nose by a flexible (rubber) strap or hinged connector instead of the single-piece support member (see FIG. 5). Elastic straps extend backward around the head to hold the device in place. Pieces of foam on the inner surface of the support members hold the device in the proper position across the bridge of the nose, and the outermost edges of the foam press on the inner ends of the eyelids to close them.

In practice, the apparatus of this invention must be “customized” to be best suited to the Subject. The support member would be selected from a variety of different sizes prepared to fit a variety of Subjects with different sizes and shapes of facial features. The design can also differ from that described in the figures, as long as it employs the method of this invention. In one embodiment, a second set of pressure protrusions that contact the lower eyelid can be added, although just pulling the upper eyelid inwardly usually closes the eye effectively.

In another embodiment, the present apparatus can be made in the form of sleep goggles, with a rigid shield over the eye socket to protect the eye from contact with bedding. Conventional sleep goggles can be converted to function as the apparatus claimed in this application by affixing suitable protrusions to the rigid lens of the goggle, e.g. as with adhesive. The apparatus of the present invention can be made to close only a single eye by putting pressure protrusions over only one eye. One skilled in the art can devise many ways to apply the method of this application to close the eyelids of Subjects.

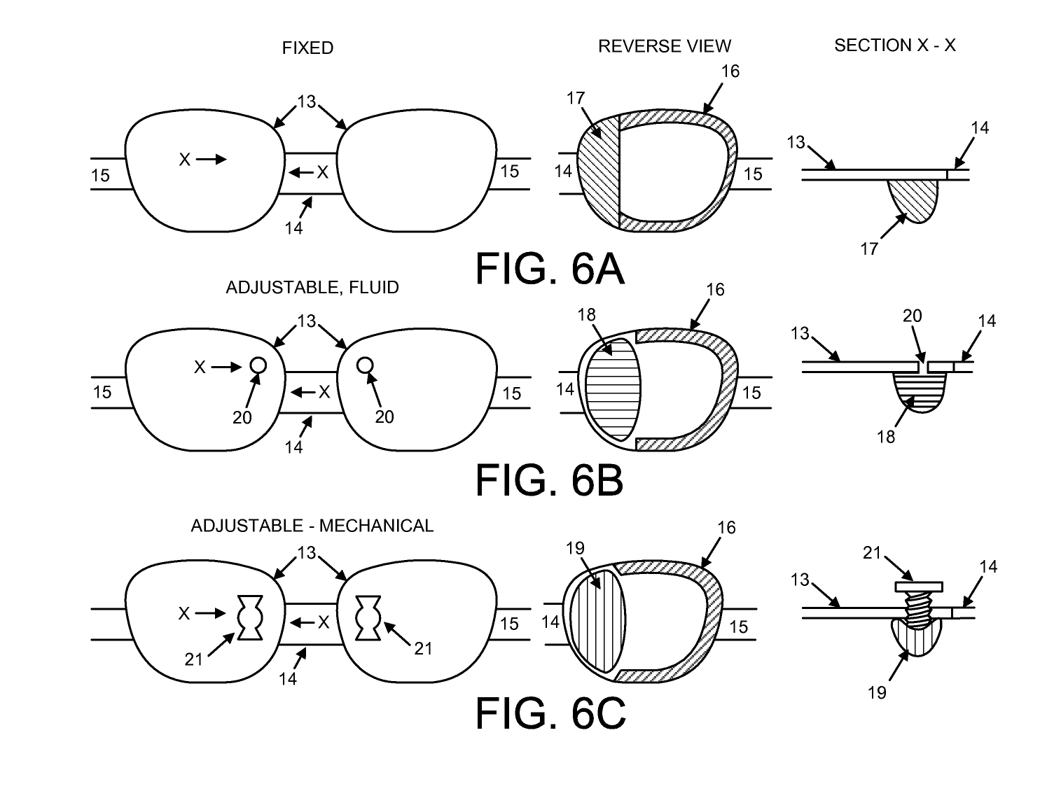

FIG. 6 illustrates designs that are modifications to existing “moisture chamber” sleep goggles like those described in the Davison 922 patent. Such “moisture chamber” sleep goggles are held over the eyes by an elastic band around the head. They have a strip of foam around the edge of each goggle where they rest on the bones around the eye socket. This foam supports the goggle on the face and forms an airtight chamber over each eye. The foam does not touch the eyelid.

In FIG. 6, goggles have foam around the edges to support them comfortably on the face, but the inner ends of the goggles (the sides touching an Subject's face) are modified to apply pressure on the inner ends of the eyelids to close the eyelids and hold them shut. In one embodiment, the foam at the inner ends of the goggles is enlarged and shaped so that it presses on the eyelids to close them when the goggles are put on (see FIG. 6A, 17). In another embodiment, FIGS. 6B and 6C show means for adjusting the size and/or shape of the contacting element that presses on the eyelids; that is, they can be extended to close the eyelids after the device is in place or can be adjusted to make the device more comfortable. For example, FIG. 6B shows an air-filled bubble or sac (18) is used to press on the eyelids to close them. In the example shown, the bubble is inflated by injecting air through a flap valve (20) similar to those used to inflate athletic balls. A hypodermic style pump or other type can be used. In one embodiment, fluids other than air can also be used.

In FIG. 6C, fluid-filled bubbles or foam protrusions are used. They are extended or adjusted by “squeezing” them mechanically in one direction to make them expand in another direction. The screw (21) shown in FIG. 6C can be turned in to make the underlying bubble or foam press against the eyelid, or out, to relieve the pressure on the eyelid. Sliding mechanisms that force the bubble or foam posteriorly can be used to accomplish the same purpose as the screw.

In one embodiment, non-adjustable contacting elements, like those in FIG. 6A, can be made protrusive by fashioning them from “memory foam”, which the user can compress just before putting the goggles on. The foam will then expand and press the user's eyelids shut as it returns to its original shape.

In one embodiment, the present invention provides an apparatus for pulling one or both eyelids over the eyes of a subject, comprising (1) a support member for positioning the apparatus on the subject's face, said support member comprises (i) an outer layer of rigid material to provide structural strength, (ii) an inner layer of soft material that contacts the subject's face, and (iii) means to properly pull the eyelids closed; and (2) a means for securing the support member on the subject's face. For example, an elastic band can be connected to the support member, wherein said elastic band can be extended around the subject's head.

In one embodiment, said means for pulling the eyelids closed comprises one or more of first pressure protrusions extending from the inner layer of the support member, wherein when the support member is secured on the subject's face, the protrusions will contact the inner portion of the subject's upper eyelid(s) and apply gentle pressure obliquely to pull the eyelid(s) towards the nose, thereby pulling the eyelid(s) over the eye(s) and holding the eye(s) closed.

In one embodiment, the pressure protrusions contact the inner ⅛ to ¼ of the length of each upper eyelid. In another embodiment, the pressure protrusion is fashioned from a soft pliable material. In one embodiment, the soft pliable material is water resistant.

In one embodiment, the support member rests on the bones at the inner ends of the subject's eye sockets above and below each eye and partially covers the subject's eyes and nose. In one embodiment, the support member comprises two support points on the forehead at about the inner ends of the eyebrows above the inner corners of the eye sockets, and two support points on either side of the nose below the inner corners of the eye sockets.

In one embodiment, the support member is fashioned in a shape comprising six arms that cross over the bridge of the subject's nose, the ends of two of said arms comprises means to pull the eyelids closed.

In one embodiment, the apparatus comprises two sections of support members that are connected across the nose by a flexible strap or hinged connector.

In one embodiment, the apparatus comprises (i) a support member fashioned in the shape of an “X” with no edges extending over the eyes, and (ii) a bar connected to the support member, wherein said bar crosses over the bridge of the nose and extends outward over the inner end of the eyes, and each end of said bar comprises the means to pull the eyelids closed.

In one embodiment, the apparatus further comprises one or more of second pressure protrusions, wherein when the support member is secured on the subject's face, the second pressure protrusions will contact the inner portion of the subject's lower eyelid.

The present invention also provides a method of closing one or both eyes of a subject, comprising the step of securing the apparatus described herein over the face of said subject. In one embodiment, the subject is a person with nocturnal lagophthalmos.

The present invention also provides an apparatus for pulling one or both eyelids over the eyes of a subject, said apparatus comprises a pair of goggles and means to pull the eyelids over the eyes, wherein said means for pulling the eyelids is located on the side of the google that touches the subject's face, wherein when said goggle is secured on the subject's face, said means to pull the eyelids will contact the inner portion of the subject's upper eyelid(s) and apply gentle pressure obliquely to pull the eyelid(s) towards the nose, thereby pulling the eyelid(s) over the eye(s) and holding the eye(s) closed. In one embodiment, the means to pull the eyelids comprises air-filled or fluid-filled sac. In one embodiment, there is also provided a method of using such apparatus to close one or both eyes of a subject, comprising the step of securing the apparatus described herein over the face of said subject. In one embodiment, the subject is a person with nocturnal lagophthalmos.

Throughout this application, various references or publications are cited. Disclosures of these references or publications in their entireties are hereby incorporated by reference into this application in order to more fully describe the state of the art to which this invention pertains.

It will be understood that the foregoing description is of preferred exemplary embodiments of the invention, and that the invention is not limited to the specific forms shown. Other modifications may be made in the design and arrangement of the elements described herein without departing from the scope of the invention as expressed in the appended claims.

What is claimed is:

1. An apparatus for pulling one or both eyelids over the eyes of a subject, comprising
a support member for positioning the apparatus on the subject's face, said support member comprises (i) an outer layer of rigid material to provide structural strength, (ii) an inner layer of soft material that contacts the subject's face, and (iii) means to properly pull the eyelids closed; and
a means for securing the support member on the subject's face.

2. The apparatus of claim 1, wherein said means for securing the support member on the subject's face is an elastic band.

3. The apparatus of claim 1, wherein said means for pulling the eyelids closed comprises one or more of first pressure protrusions extending from the inner layer of the support member, wherein when the support member is secured on the subject's face, the protrusions will contact the inner portion of the subject's upper eyelid(s) and apply gentle pressure obliquely to pull the eyelid(s) towards the nose, thereby pulling the eyelid(s) over the eye(s) and holding the eye(s) closed.

4. The apparatus of claim 3, wherein the pressure protrusions contact the inner ⅛ to ¼ of the length of each upper eyelid.

5. The apparatus of claim 3, wherein the pressure protrusion is fashioned from a soft pliable material.

6. The apparatus of claim 4, wherein the soft pliable material is water resistant.

7. The apparatus of claim 1, wherein said support member rests on the bones at the inner ends of the subject's eye sockets above and below each eye and partially covers the subject's eyes and nose.

8. The apparatus of claim 1, wherein the support member comprises two support points on the forehead at about the inner ends of the eyebrows above the inner corners of the eye sockets, and two support points on either side of the nose below the inner corners of the eye sockets.

9. The apparatus of claim 1, wherein the support member is fashioned in a shape comprising six arms that cross over the bridge of the subject's nose, the ends of two of said arms comprises means to pull the eyelids closed.

10. The apparatus of claim 1, wherein the apparatus comprises two sections of support members that are connected across the nose by a flexible strap or hinged connector.

11. The apparatus of claim 1, wherein the apparatus comprises (i) a support member fashioned in the shape of an “X” with no edges extending over the eyes, and (ii) a bar connected to the support member, wherein said bar crosses over the bridge of the nose and extends outward over the inner end of the eyes, and each end of said bar comprises means to pull the eyelids closed.

12. The apparatus of claim 1, wherein the apparatus further comprises one or more of second pressure protrusions, wherein when the support member is secured on the subject's face, the second pressure protrusions will contact the inner portion of the subject's lower eyelid.

13. A method of closing one or both eyes of a subject, comprising the step of securing the apparatus of claim 1 over the face of said subject.

14. The method of claim 13, wherein the subject is a person with nocturnal lagophthalmos.

15. An apparatus for pulling one or both eyelids over the eyes of a subject, said apparatus comprises a pair of goggle and means to pull the eyelids over the eyes, wherein said means for pulling the eyelids is located on the side of the google that touches the subject's face, wherein when said goggle is secured on the subject's face, said means to pull the eyelids will contact the inner portion of the subject's upper eyelid(s) and apply gentle pressure obliquely to pull the eyelid(s) towards the nose, thereby pulling the eyelid(s) over the eye(s) and holding the eye(s) closed.

16. The apparatus of claim 15, wherein said means to pull the eyelids comprises air-filled or fluid-filled sac.

17. A method of closing one or both eyes of a subject, comprising the step of securing the apparatus of claim 15 over the face of said subject.

18. The method of claim 17, wherein the subject is a person with nocturnal lagophthalmos.
